(19) United States
(12) Patent Application Publication
(10) Pub. No.: US 2005/0112149 A1
(43) Pub. Date: May 26, 2005

(54) Single-dose taste inhibitor units

(76) Inventors: W. Michael Belote, Dublin, GA (US); Connie Belote, Dublin, GA (US); Billie Pitts, Dublin, GA (US)

Correspondence Address: MEHRMAN LAW OFFICE, P.C.;One Premier Plaza, Suite 795, 5605 Glenridge Drive, Atlanta, GA, 30342, US

(21) Appl. No.: 10/977,805

(22) Filed: Oct. 29, 2004

Related U.S. Application Data
(60) Provisional application No. 60/515,805, filed on Oct. 29, 2003.

(51) Int. Cl. A61K 38/17; A61K 9/68; A61K 7/16; A61K 35/78; A61K 9/00

(52) U.S. Cl. 424/400; 424/760; 514/12; 424/49; 424/440; 424/48

(57) ABSTRACT

A package of single-dose taste inhibitor units available in multiple forms, such as lozenges, dissolvable strips, gum, chewable tablets, hard candies, and candies or chewing gums with liquid or gel centers. They may also come in liquid forms such as a mouthwash, rinse or spray which may be administered by means of a pump, aerosol spray or cup. The taste inhibitor agents contained in the taste inhibitor units operate to mask or reduce the flavor of foods and/or anesthetize the taste buds thereby diminishing the desire for food intake.

### REFERENCE TO RELATED APPLICATION

This application claims the benefit of the filing date of U.S. Provisional Patent Application No. 60/515,805 filed on Oct. 10, 2003.

### TECHNICAL FIELD

The present invention relates to a taste inhibitor that an individual can ingest orally, on demand, to quell his or her appetite and thereby reduce food intake.

### BACKGROUND

The battle of the waistline bulge continues with a vengeance. Yet there remains an unmet need for simple, inexpensive, and perhaps most importantly, easy ways to lose weight and keep it off. Some people have even gone to the extreme of having major surgery of have their stomachs stapled down to the size of a avocado. There has to be a better, safer and less expensive way to go about solving this problem.

### SUMMARY OF THE INVENTION

The present invention solves this problem in a simple, inexpensive, and easy-to-use taste inhibitor that anyone can take “on the spot,” for example after eating a reasonable helping of a favorite meal or snack, to take away the enjoyment of the food and thereby quell the appetite. The basic idea is to have taste inhibitor units, such as dissolvable strips, lozenges or chewing gum, readily available for consumption during a meal. The taste inhibitor units should be packaged as single-dose units carried in a convenient size package, for example similar to the pack of gum or a cartridge of pull-out breath freshening strips. In this manner, the individual can take a taste inhibitor unit after eating a reasonable portion of a meal or snack, and thereby break the craving and stop the desire to continue eating. For example, after five spoonfuls of ice cream, the individual might ingest a taste inhibitor unit in order to avoid eating the entire carton.

The taste inhibitor units will be particularly helpful with binge foods that some people tend to consume well beyond reason, such as chocolate, ice cream, Cheetos®, Bon-Bons®, and so forth. The taste inhibitor units may also be helpful in connection with traditional meals for persons who want to limit their intake below previously accustomed levels. In view of the extreme measures some people are willing to go to in order to reduce their food consumption, such as surgical removal or stapling part of the stomach, the taste inhibitor units present a valuable and safe alternative. The taste inhibitor units may also have a helpful psychological effect for people with eating disorders such as anorexia nervosa and bulimia. In this regard, it is anticipated that the anxiety associated with eating experienced by at least some people suffering from these eating disorders will be relieved by the knowledge that the taste inhibitor units are available to help them stop eating once they get started, and this added sense of security will enable them to eat more normally.

In view of the foregoing, it will be appreciated that the single-dose taste inhibitor unit of the present invention represents a significantly and much needed improvement for weight control. The specific techniques and structures to implement the invention will become apparent from the following detailed description of the embodiments of the invention and the appended drawings and claims.

### BRIEF DESCRIPTION OF THE DRAWINGS

FIG. 1 shows a single-dose taste inhibitor unit configured as a stick of chewing gum.

FIG. 2 shows a single-dose taste inhibitor unit configured a lozenge.

FIG. 3 shows a single-dose taste inhibitor unit configured a dissolvable strip and a cartridge of such strips.

### DETAILED DESCRIPTION OF THE PREFERRED EMBODIMENTS

The taste inhibitor units may be made available in multiple forms, such as lozenges, dissolvable strips, gum, chewable tablets, hard candies, and candies or chewing gums with liquid or gel centers. They may also come in liquid forms such as a mouthwash, rinse or spray which may be administered by means of a pump, aerosol spray or cup. The inhibitor agents contained in the taste inhibitor units operate to mask or reduce the flavor of foods and/or anesthetize the taste buds thereby diminishing the desire for food intake.

The taste inhibitor units should be pleasant looking, pleasant tasting and, in certain forms such as dissolvable strips, completely dissolve upon contact with moisture within the mouth. Although taste inhibitor agents added to a chewing gum or lozenge will dissolve more slowly, any form that successfully delivers the taste inhibitor agents may be used. In fact, gum or chewable tablets with liquid or gel centers carrying the taste inhibitor agents may be the most palatable and successful delivery forms for many individuals.

Dietary aids are neither a new nor uncommon means to suppress appetites and are regularly used by individuals wishing to fight obesity. However, most of the products available to the general consumer are in pill form and are typically taken orally with a full glass of water, and are not immediately effective. For this reason, conventional appetite suppressants must be taken well before a meal, and effect the desire for and enjoyment of the entire meal, rather than just the portion after a desired intake as with the present taste inhibitor units.

In addition, the potency of most medications ingested in pill form may be diminished in the gastrointestinal tract, thereby reducing the effectiveness of the medication. However, these same active ingredients may also be safely absorbed through the buccal linings of the mouth by ingesting the medication in powder form. Typically, the medication dissolves after it is deposited on the tongue. Individuals using this means of ingestion will generally experience a bitter taste and may have problems maintaining the medication in the mouth cavity long enough for absorption to take place. Again, some of the potency of the active ingredients may be lost if not adequately absorbed.

Administering the taste bud inhibitors by means of a tasty lozenge, dissolvable strip, chewing gum or spray will allow the active ingredients to safely absorb through the buccal lining of the mouth and preserve the potency of the taste inhibitor. The active ingredients delivered by the taste inhibitor units may include, but are not limited to the following agents:
Leptin—a protein hormone, acts as a sweet-sensing modulator and regulates food intake, body fat and energy loss;
Gurmarin (Gymnema sylvestre)—an herbal extract sometimes used to anesthetize the sweet taste buds and induce weight loss. Commonly used in combination with the herb, Blue Flag; and
Capsaicin—found in the seeds and membranes of a chili pepper, sometimes used to reduce the intensity of certain taste qualities.

Inactive or sweetening ingredients used for the taste inhibitor units may include but are not limited to the following agents:
Maltodextrin—made from natural corn starch, it is generally used as a source of energy and is highly digestible;
Carrageenan Gum—red seaweed, a thickening agent typically used for chewing gum, will assist in entrapping the inhibitor agents, thereby allowing the active ingredients to release in a prolonged and controlled fashion;
Humectant Glycerine—A sweetener sometimes used as a solvent and preservative for foods;
Saccharin—Calorie-free sweetener;
Ascorbic Acid: Vitamin C;
Potassium Acesulfame—Low calorie sweetener; and
Locust Bean Gum—A thickener with high viscosity

The following active ingredients may also be used: Chromium, Vanadium, Benzocaine, Caffeine and Green Tea Extract.

Of course, the invention is not limited to these particular ingredients, and may be practiced with other taste inhibitor agents and delivery agents presently known or developed in the future. The important point is that that the taste inhibitor units can be readily delivered directly to the mouth to have a substantially immediate effect. This allows the taste inhibitor units to be taken conveniently on-the-spot, preferably during the course of a meal or other eating event, and thereby alter the individual from his or her usual eating habits.

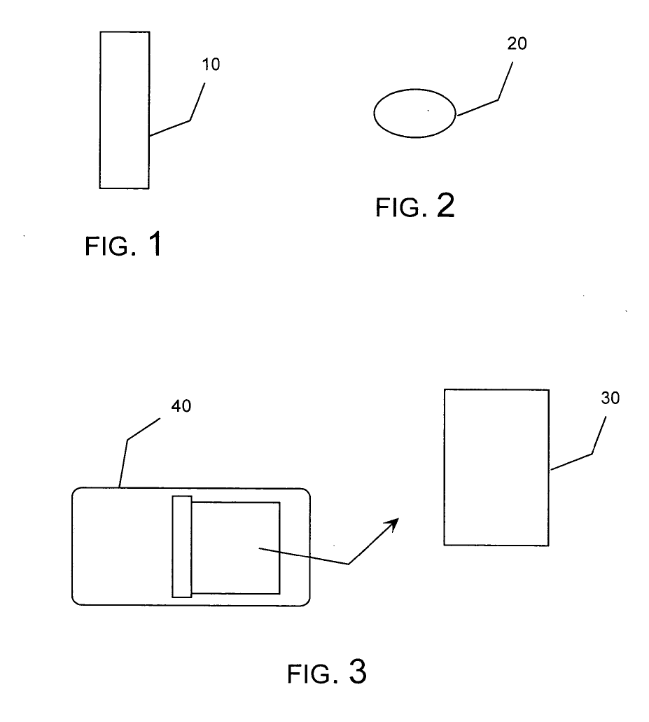

FIG. 1 shows a single-dose taste inhibitor unit 10 configured as a stick of chewing gum. This type of taste inhibitor unit would typically be package like a traditional pack of gum containing a number such as 5 or 20 sticks of gum.

FIG. 2 shows a single-dose taste inhibitor unit 20 configured a lozenge. This type of taste inhibitor unit would typically be package like a traditional package of lozenges, for example in a zip-lock bag containing 20 or 30 lozenges.

FIG. 3 shows a single-dose taste inhibitor unit 30 configured a dissolvable strip and a cartridge 40 of such strips. This type of taste inhibitor unit would typically be package like a traditional package of dissolvable strip, for example in a cartridge 40 containing 20 or 30 dissolvable strips.

In view of the foregoing, it will be appreciated that present invention provides significant improvements for easy, inexpensive and safe weight control. It should be understood that the foregoing relates only to the exemplary embodiments of the present invention, and that numerous changes may be made therein without departing from the spirit and scope of the invention as defined by the following claims.

1. A taste inhibitor comprising a single-dose taste inhibitor unit configured for ingestion by an individual for the purpose of masking or reducing the flavor of food and thereby quelling the individual's appetite.

2. The taste inhibitor of claim 1, wherein the single-dose taste inhibitor unit is selected from the group consisting of lozenges, dissolvable strips, gum, chewable tablets, hard candies, and candies or chewing gums with liquid or gel centers.

3. The taste inhibitor of claim 1, wherein the single-dose taste inhibitor unit comprises a liquid form selected from the group consisting of mouthwash, rinse, spray.

4. The taste inhibitor of claim 3, wherein the liquid is administered by a pump, aerosol spray or cup.

5. The taste inhibitor of claim 1, comprising an active ingredient selected from the group consisting of Leptin, Gurmarin and Capsaicin.

6. The taste inhibitor of claim 5, further comprising an inactive or sweetening ingredient selected from the group consisting of Maltodextrin, Carrageenan Gum; Humectant Glycerine, Saccharin; Potassium Acesulfame and Locust Bean Gum.

7. The taste inhibitor of claim 1, comprising an active ingredient selected from the group consisting of Chromium, Vanadium, Benzocaine, Caffeine and Green Tea Extract.

8. The taste inhibitor of claim 7, further comprising an inactive or sweetening ingredient selected from the group consisting of Maltodextrin, Carrageenan Gum; Humectant Glycerine, Saccharin; Potassium Acesulfame and Locust Bean Gum.

9. A taste inhibitor comprising a package of single-dose taste inhibitor units configured for ingestion by an individual for the purpose of masking or reducing the flavor of food and thereby quelling the individual's appetite.

10. The taste inhibitor of claim 9, wherein the single-dose taste inhibitor units are selected from the group consisting of lozenges, dissolvable strips, gum, chewable tablets, hard candies, and candies or chewing gums with liquid or gel centers.

11. The taste inhibitor of claim 9, wherein the single-dose taste inhibitor units comprises a liquid form selected from the group consisting of mouthwash, rinse, spray.

12. The taste inhibitor of claim 11, wherein the liquid is administered by a pump, aerosol spray or cup.

13. The taste inhibitor of claim 9, comprising an active ingredient selected from the group consisting of Leptin, Gurmarin and Capsaicin.

14. The taste inhibitor of claim 13, further comprising an inactive or sweetening ingredient selected from the group consisting of Maltodextrin, Carrageenan Gum; Humectant Glycerine, Saccharin; Potassium Acesulfame and Locust Bean Gum.

15. The taste inhibitor of claim 9, comprising an active ingredient selected from the group consisting of Chromium, Vanadium, Benzocaine, Caffeine and Green Tea Extract.

16. The taste inhibitor of claim 15, further comprising an inactive or sweetening ingredient selected from the group consisting of Maltodextrin, Carrageenan Gum; Humectant Glycerine, Saccharin; Potassium Acesulfame and Locust Bean Gum.

17. A method for weight control, comprising the steps of:
obtaining a package of single-dose taste inhibitor units configured for ingestion by for the purpose of masking or reducing the flavor of food and thereby quelling the individual's appetite;
consuming a portion of a meal or snack;
ingesting at least one of the taste inhibitor units; and
discontinuing consumption of the meal or snack as assisted by the taste inhibitor unit.

18. The method of claim 17, further comprising the step of repeating the method of claim 17 in compliance with a regimen designed to cause weight loss.

19. The method of claim 17, further comprising the step of repeating the method of claim 17 in compliance with a regimen designed to maintain weight.

20. The method of claim 17, wherein the package of single-dose taste inhibitor units is selected from the group consisting of lozenges, dissolvable strips, gum, chewable tablets, hard candies, candies or chewing gums with liquid or gel centers, mouthwash, rinse and spray.
